(19) United States
(12) Patent Application Publication
(10) Pub. No.: US 2005/0131077 A1
(43) Pub. Date: Jun. 16, 2005

(54) Preventing or reducing the adhesion of microorganisms with phytanetriol

(75) Inventor: Anthony Potin, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/979,235

(22) Filed: Nov. 3, 2004

Related U.S. Application Data
(60) Provisional application No. 60/526,991, filed on Dec. 5, 2003.

(30) Foreign Application Priority Data
Nov. 7, 2003 (FR) 0350809

(51) Int. Cl. A61K 31/045

(52) U.S. Cl. 514/724

(57) ABSTRACT

The present invention relates to the use of phytanetriol (3,7,11,15-tetramethyl-1,2,3-hexadecanetriol) as an agent for preventing or reducing the adhesion of microorganisms to the surface of the skin and/or mucous membranes.

### REFERENCE TO PRIOR APPLICATIONS

This application claims priority to U.S. provisional application 60/526,991 filed Dec. 5, 2003, and to French patent application 0350809 filed Nov. 7, 2003, both incorporated herein by reference.

### FIELD OF THE INVENTION

The present invention relates to the use of phytanetriol as an agent for preventing or reducing the adhesion of microorganisms to the surface of the skin and/or mucous membranes. The invention also relates to the preparation of a cosmetic and/or dermatological composition comprising phytanetriol.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Human skin is permanently populated with a multitude of different microorganisms (bacteria, yeasts and fungi). The commensal microorganisms living on or in the skin may form part of a microflora that is either resident (normal) or transient. The resident microbial flora, which is essential for good health of the skin, consists mainly of staphylococci (Staphylococcus epidermis and Staphylococcus hominis), corynebacteria, Gram+propionibacteria such as Propionibacterium acnes, and also a fungal flora mainly composed of Pytosporum ovale. They are present in well-defined distribution profiles. Usually, the transient microorganisms do not become solidly attached; they are incapable of multiplying, and normally die within a few hours.

The anatomy and physiology of the skin vary from one part of the body to another, and the resident microflora reflects these variations.

Most of the skin bacteria are present on the superficial squamous epidermis, colonizing dead cells, and closely associated with the sebaceous and sweat glands. The excretions from these glands provide water, amino acids, urea, electrolytes and specific fatty acids serving as nutrient elements mainly for Staphylococcus epidermidis and certain aerobic corynebacteria.

Skin infections are usually caused by disruption of the ecological equilibrium of the resident flora following colonization of the skin by pathogenic exogenous germs or following abnormal proliferation of an endogenous strain. The pathogenic germs that are the most common are Pseudomonas aeruginosa (Gram−), which is responsible for small spots, folliculitis, red patches and pruritus, Candida albicans, which can cause inflammation at the labial angle, cutaneous candidiasis, pruritus, folliculitis and aphthae, Staphylococcus aureus, which can cause spots, folliculitis, impetigo and furuncles, and Group A Streptococcus, responsible for impetigo.

It is common practice to use antibiotics or bactericides to combat these microorganisms. However, the use of these compounds poses the problem of the non-specificity of action targeting, without distinction, on the pathogenic flora and the resident flora, the problem of the risk of appearance of bacterial resistance, and also problems of skin tolerance (irritation, allergies, etc.).

It is also known practice to reduce or prevent the colonization of surfaces such as the teeth, the skin and/or mucous membranes, by pathogenic germs, by preventing their attachment to these supports. The compounds used as antiadhesion agents described in the prior art are either silicones (WO 99/62475) or carbohydrates and carbohydrate derivatives, such as those described in patent application WO 96/23479, or plant oils, as described in patent application EP-1 133 979.

However, most carbohydrates constitute a source of carbon for bacteria and fungi. Their presence in cosmetic compositions consequently promotes microbial proliferation and necessitates an increase in the concentration of preserving agents (bactericides or bacteriostatic agents). This drawback thus negates the benefit of the approach consisting in replacing antibiotic compounds or bactericides with compounds that reduce microbial adhesion.

### DETAILED DESCRIPTION OF THE PREFERRED EMBODIMENTS

The inventor has demonstrated, surprisingly, that phytanetriol makes it possible to significantly reduce the adhesion of microorganisms to the surface of the skin and/or mucous membranes, and thus to prevent the proliferation of potentially pathogenic germs in the absence of antibiotic, bactericidal or fungicidal agents, without it being necessary to use a large amount of preserving agents.

Phytanetriol, or 3,7,11,15-tetramethyl-1,2,3-hexadecanetriol, is a known compound, which is marketed for example under the name “Phytanetriol-63926” by the company Roche. It has been used in cosmetic compositions, especially in combination with farnesol and at least one other active agent in order to regulate visible or tactile skin discontinuities (WO 2000/062745). It has also been described in patent application EP-A-1 161 938 as an agent capable of limiting the penetration of pollutants into the skin, thus protecting it against the deleterious effects of the pollution.

While bound by no particular theory, phytanetriol is believed to act on the physicochemical properties of the surface of the skin and/or mucous membranes, these physicochemical properties involving electrodynamic interactions due to the Van der Waals forces, the Lewis acid-base interactions and electrostatic interactions. This is unlike carbohydrates, which bind to the receptors of microorganisms to prevent binding to the glycolipids of corneocytes.

One subject of the invention is thus the use of phytanetriol, optionally in a composition, as an agent for preventing or reducing the adhesion of microorganisms to the surface of the skin and/or mucous membranes.

Another subject of the invention is also the use of phytanetriol for preventing or combating pathologies associated with the adhesion of microorganisms to the surface of the skin and/or mucous membranes.

The expression “preventing or reducing the adhesion of microorganisms” should be understood as meaning that phytanetriol or the composition containing it may be used either preventively, for its ability to totally or partially prevent the adhesion of microorganisms, or curatively, for its ability to facilitate the detachment of microorganisms.

The phytanetriol may be present in the invention composition for topical application in an amount ranging, for example, from 0.001% to 20% by weight and preferably from 0.1% to 10% by weight relative to the total weight of the composition.

The phytanetriol used according to the invention may be incorporated especially into compositions that may in particular be in the form of oil-in-water (O/W) or water-in-oil (W/O) emulsions or in the form of cubic gel particles, these particles possibly being used alone or incorporated in an emulsion.

The term “cubic gel” denotes transparent gels which are isotropic in polarized light and which are in the form of a cubic liquid crystal phase. The cubic phases are organized in a bipolar manner into distinct hydrophilic and lipophilic domains, in close contact and forming a thermodynamically stable three-dimensional network. Such an organization has been disclosed in particular in Luzzati (1968), “Biological Membranes” (Chapman, D. Ed.), vol. 1, 71-123 and in Mariani et al. (1988), J. Mol. Biol., 204, 165-189, and also in “La Recherche” (1992), vol. 23, 306-315. According to the arrangement of the hydrophilic and lipophilic domains, the cubic phase is said to be of normal or inverse type. The term “cubic gel” used according to the present invention combines, of course, gels with cubic phases of different types.

When the cubic gel is dispersed in an aqueous medium, particles of cubic gel in dispersion are obtained, these particles having the same structure as the undispersed cubic gel.

The cubic gel particles containing phytanetriol may be present in the topical-application composition used according to the invention in an amount ranging, for example, from 0.1% to 20% by weight and preferably from 0.1% to 10% by weight relative to the total weight of the composition.

The cubic gel particles containing phytanetriol are advantageously in aqueous dispersion in the composition. These particles may be obtained in particular by the preferred embodiment described below.

According to this embodiment, the particles are in aqueous dispersion and are essentially formed by a mixture comprising (i) 0.1% to 15% by weight, relative to the total weight of the composition, of 3,7,11,15-tetramethyl-1,2,3-hexadecanetriol or phytanetriol, optionally combined with an N-2-alkoxycarbonyl N-methylglucamine derivative and/or an unsaturated fatty acid monoglyceride, and (ii) 0.05% to 3% by weight, relative to the total weight of the composition, of at least one dispersing and stabilizing agent, the said agent being selected from the group consisting of surfactants that are water-soluble at room temperature, containing a saturated or unsaturated, linear or branched fatty chain containing from 8 to 22 carbon atoms. The percentages are expressed herein relative to the total amount of the composition containing the phytanetriol-based cubic gel particles.

According to this embodiment of the cubic gel particles used according to the invention, the relative weight proportion of phytanetriol relative to the weight of the dispersing and stabilizing agent (ii) may range, for example, from 2 to 200 and is preferably less than or equal to 50 (in particular ranging from 2 to 50).

Among the N-2-alkoxycarbonyl N-methylglucamine derivatives, mention may be made in particular of those corresponding to formula (I) below: 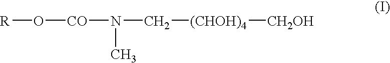
in which R represents a branched alkyl radical containing from 6 to 18 carbon atoms.

Among these derivatives, mention may be made in particular of N-2-hexyldecyloxycarbonyl-N-methylglucamine, N-2-ethylhexyloxycarbonyl-N-methylglucamine and N-2-butyloctyloxy-carbonyl-N-methylglucamine, and mixtures thereof.

The compounds of formula (I) as defined above are disclosed and may be prepared according to the process disclosed in document EP-A-711 540, which is cited herein for reference. This process in particular comprises the steps consisting in:
(a) dissolving N-methylglucamine in a mixture of water and an organic solvent, the solvent possibly being tetrahydrofuran, for example,
(b) dispersing sodium bicarbonate in the mixture obtained above, in a suitable proportion corresponding to about four times the molar proportion of N-methylglucamine,
(c) then introducing an alkyl chloroformate, the alkyl radical being C₆-C₁₈, into the reaction mixture obtained, in a suitable proportion, generally an equimolar proportion relative to that of N-methylglucamine, and then leaving the mixture to react, and
(d) filtering the reaction mixture obtained after step (c), collecting the pasty residue obtained by filtration and then dissolving it in acetone to crystallize it at a temperature of about 5° C. After filtration, the crystals of the N-2-alkoxycarbonyl N-methylglucamine derivative formed are spin-filtered and dried under vacuum.

When phytanetriol is used as a mixture with one or more compounds of formula (I), this mixture preferably comprises an amount of phytanetriol ranging from 1% to 40% by weight and better still from 10% to 30% by weight relative to the weight of the mixture, and an amount of N-2-alkoxycarbonyl N-methylglucamine derivative of formula (I) ranging from 60% to 99% by weight and better still from 70% to 90% by weight relative to the weight of the mixture.

The unsaturated fatty acid monoglycerides that may be used as a mixture with phytanetriol to prepare cubic gel particles are preferably those with an unsaturated fatty chain containing from 16 to 22 carbon atoms. Among these monoglycerides, mention may be made in particular of glyceryl monooleate or monoolein and glyceryl monolinoleate or monolinolein. Needless to say, to prepare the dispersions of cubic gel particles, it is possible to use a mixture of monoglycerides as defined above, and also a mixture of unsaturated fatty acid monoglycerides and of saturated fatty acid monoglycerides, the proportion of saturated fatty acid monoglycerides however preferably being less than that of the unsaturated fatty acid monoglycerides.

When phytanetriol is used as a mixture with unsaturated fatty acid monoglycerides, this mixture preferably comprises an amount of phytanetriol ranging from 1% to 50% by weight and better still from 10% to 30% by weight relative to the total weight of the mixture and an amount of unsaturated fatty acid monoglyceride in a proportion of from 50% to 99% by weight and better still from 70% to 90% by weight relative to the weight of the mixture.

The agent (ii) for dispersing and stabilizing the cubic gel particles is preferably selected from the group consisting of:
(1) alkyl or alkenyl ethers or esters of a polyol,
(2) N-acyl amino acids and derivatives thereof, and peptides N-acylated with an alkyl or alkenyl radical, and salts thereof,
(3) alkyl or alkenyl ether or ester sulfates, derivatives thereof and salts thereof,
(4) polyoxyethylenated fatty alkyl or alkenyl ethers or esters,
(5) polyoxyethylenated alkyl or alkenyl carboxylic acids and salts thereof,
(6) N-alkyl or alkenyl betaines,
   (7) alkyl or alkenyl trimethylammoniums and salts thereof, and
(8) mixtures thereof.

In the compounds listed above, the alkyl and alkenyl radicals preferably contain from 8 to 22 carbon atoms and may be in the form of mixtures.

(1) Useful alkyl or alkenyl ethers or esters of a polyol include:
(a) sorbitan alkyl or alkenyl esters polyoxyethylenated with at least 20 ethylene oxide units, such as sorbitan palmitate 20 EO or Polysorbate 40 sold under the name “Montanox 40 DF” by the company SEPPIC, and sorbitan laurate 20 EO or Polysorbate 20 sold under the name “Tween 20” by the company ICI,
(b) oxyethylenated or non-oxyethylenated polyglyceryl alkyl or alkenyl esters comprising at least 10 units derived from glycerol, such as polyglyceryl-10 laurate sold under the name “Decaglyn 1-L” by the company Nikko Chemicals,
(c) polyglyceryl alkyl or alkenyl ethers, such as polyglyceryl-3 hydroxylauryl ether sold under the name “Chimexane NF” by the company Chimex, and
(d) alkyl or alkenyl esters or ethers of mono- or polysaccharides, such as those derived from glucose, fructose, galactose, maltose or lactose, and in particular 1- and 6-monoesters of D-fructose, of decylglucose and of decylpolyglucose.

(2) As N-acyl amino acids and derivatives thereof, and peptides N-acylated with an alkyl or alkenyl radical, and salts thereof, the ones that are preferably used are those for which the alkyl or alkenyl radical contains at least 12 carbon atoms.

According to the invention, the term “amino acids” means alpha-, beta- or gamma-amino acids. N-Acylamino acid salts that may be mentioned, for example, are those of N-acylglutamate, such as monosodium cocoylglutamate, monosodium lauroylglutamate, disodium (C₁₄-C₂₀)alkylglutamate (the C₁₄-C₂₀ alkyl radical being derived from hydrogenated tallow), sold respectively under the names “Acylglutamate CS-11”, “Acylglutamate LS-11” and “Acylglutamate HS-21” by the company Ajinomoto. Mention may also be made of N-acyl lysines such as lauroyllysine sold under the name “Amihope LL” by the company Ajinomoto. The N-acyl amino acid derivatives and salts thereof are preferably N-acyl sarcosinates such as the sodium lauroylsarcosinate sold under the name “Oramix L30” by the company SEPPIC and the sodium myristoylsarcosinate and sodium palmitoylsarcosinate sold respectively under the names “Nikkol Sarcosinate MN” and “Nikkol Sarcosinate PN” by the company Nikko Chemicals.

Among the N-acyl peptides that may be mentioned are those derived from all or part of collagen or keratin, such as the sodium lauroyl collagen and palmitoyl keratin sold under the names “Proteol B 30” and “Lipacide PK” by the company SEPPIC.

(3) Among the alkyl or alkenyl ether or ester sulfates, derivatives thereof and salts thereof, the ones that are preferably used are those for which the alkyl or alkenyl radical contains at least 12 carbon atoms.

Among the alkyl or alkenyl ether sulfates, the ones that are preferably used are alkyl ether sulfate salts and in particular sodium lauryl ether sulfate. Among the alkyl or alkenyl ester sulfates that may be mentioned, for example, are isethionic acid esters and its salts, and in particular the sodium cocoyl isethionate sold under the name “Geropon AC 78” by the company Rhône-Poulenc.

(4) Among the polyoxyethylenated fatty alkyl or alkenyl ethers or esters that are preferably used are those for which the alkyl or alkenyl radical contains at least 12 carbon atoms. Those particularly preferred contain at least 20 ethylene oxide units, such as, for example, PEG-20 stearate, laureth-23, oleth-20 and PEG-25 phytosterol.

(5) Among the polyoxyethylenated alkyl or alkenyl carboxylic acids and salts thereof which are preferably used are those comprising at least 10 ethylene oxide units, such as, for example, laureth-10 carboxylic acid and oleth-10 carboxylic acid.

(6) Among the N-alkyl or alkenyl betaines that are preferably used are those for which the alkyl or alkenyl radical contains at least 12 carbon atoms, such as, for example, laurylamidopropylbetaine and oleylamidopropylbetaine.

(7) Among the alkyl or alkenyl trimethylammoniums and salts thereof that are preferably used are those for which the alkyl or alkenyl radical contains at least 12 carbon atoms. Salts that are preferably used are the bromides and chlorides, such as cocoyltrimethylammonium chloride and cetyltrimethylammonium bromide.

According to one particular embodiment of the invention, a water-insoluble ionic amphiphilic lipid may be added to the aqueous dispersion containing the cubic gel particles, preferably in an amount ranging from 0.0005% to 5% by weight and better still from 0.001% to 2% by weight relative to the total weight of the dispersion. Among the water-insoluble ionic amphiphilic lipids that may be mentioned in particular are:
(i) phospholipids such as natural phospholipids, for instance soya lecithin or egg lecithin, chemically or enzymatically modified phospholipids, for instance hydrogenated lecithin or the sodium salt of phosphatidic acid, and synthetic phospholipids such as dipalmitoylphosphatidylcholine,
(ii) phosphoric esters of fatty acids and salts thereof, in particular the sodium and potassium salts thereof, such as the monocetyl phosphate sold under the name “Monafax 160” by the company Mona, and the dimyristyl phosphate sold under the name “Mexoryl SY” by the company Chimex,
(iii) N-acyl derivatives of glutamic acid and salts thereof, such as the monosodium stearoylglutamate sold under the name “Acylglutamate HS 11” by the company Ajinomoto, and the mixture monosodium cocoyl/(C₁₄-C₂₀) alkyl glutamate, the C₁₄-C₂₀ alkyl radical being derived from hydrogenated tallow, sold under the name “Acylglutamate GS 11” by the company Ajinomoto,
(iv) the sodium cetyl sulfate sold under the name “Nikkol SCS” by the company Nikko Chemicals,
(v) the sodium cocoyl monoglyceride sulfate sold under the name “Nikkol SGC 80 N” by the company Nikko Chemicals, and
(vi) water-insoluble quaternary ammonium derivatives such as behenyltrimethylammonium chloride, dilauryldimethylammonium chloride, distearyldimethylammonium chloride, 4,5-dihydro-1-methyl-2-(C₁₄-C₂₀)alkyl-1-(2-(C₁₄-C₂₀)alkylaminoethyl)imidazolium methyl sulfate, the C₁₄-C₂₀ alkyl radicals being derived from hydrogenated tallow, sold under the name “Rewoquat W75H” by the company Rewo Chemische, dialkylhydroxy-ethylmethylammonium methyl sulfate whose alkyl radicals are derived from hydrogenated or unhydrogenated tallow, sold under the name “Stepanquat VP 85” by the company Stepan, and “Quaternium-82” sold by the company SEPPIC under the name “Amonyl DM”.

The incorporation of these water-insoluble ionic amphiphilic lipids gives the cubic gel particles a surface charge that results in electrostatic repulsion between the particles.

The cubic gel particles as defined above have a size which may be modified by the nature and concentration of the compounds of which they are made. These particles generally have a number-average size, measured using a BI 90 laser granulometer from the company Brookhaven Instruments Corporation, of from about 0.05 μm to about 1 μm and preferably less than or equal to 0.5 μm.

It is also possible to incorporate active compounds of various types into the cubic gel particles. In particular, the said particles may contain a hydrophilic or lipophilic active principle. Needless to say, by virtue of the specific structure of the cubic gel particles, it is possible to incorporate therein both hydrophilic and lipophilic active principles, even if these active principles are incompatible to a certain extent.

The compositions used according to the invention may in particular constitute cosmetic and dermatological compositions. For such an application, they contain a physiologically acceptable medium. The expression “physiologically acceptable medium” means herein a medium that is compatible with the skin, and where appropriate with the lips, the scalp, the eyelashes, the eyes and/or the hair. This physiologically acceptable medium may more particularly consist of water and optionally of a physiologically acceptable organic solvent chosen, for example, from lower alcohols containing from 1 to 4 carbon atoms, for instance ethanol, isopropanol, propanol or butanol; polyethylene glycols containing from 6 to 80 ethylene oxide units; polyols, for instance propylene glycol, isoprene glycol, butylene glycol, glycerol or sorbitol. The physiologically acceptable medium of the composition according to the invention has a pH which is compatible with the skin and which preferably ranges from 3 to 8 and better still from 5 to 7.

According to one preferred embodiment, the compositions used in the present invention also comprise an oily phase, which especially provides a sensation of comfort and softness when applied to the skin. The amount of oily phase may range, for example, from 2% to 40% by weight and preferably from 5% to 25% by weight relative to the total weight of the composition, the remainder of the composition consisting of the aqueous phase containing phytanetriol or composed of the cubic gel particles containing the phytanetriol, or containing the aqueous dispersion of cubic gel particles containing phytanetriol. When phytanetriol is present in cubic gel particles, the weight ratio of the amphiphilic compounds constituting the particles of the cubic phase and of the oily phase preferably ranges from 0.02/1 to 1/1 and better still from 0.05/1 to 0.5/1.

As oils which may be used in the invention, mention may be made of mineral oils (liquid petroleum jelly), oils of plant origin (liquid fraction of karite butter, sunflower oil or apricot kernel oil), oils of animal origin (perhydrosqualene), synthetic oils (hydrogenated polyisobutene, isostearyl neopentanoate or isopropyl myristate), non-volatile or volatile silicone oils (cyclomethicones such as cyclopentasiloxane and cyclohexasiloxane) and fluoro oils (perfluoropolyethers). Fatty substances that may also be used are fatty alcohols, fatty acids and waxes. The oily phase of the emulsion may also contain gums such as silicone gums, resins and waxes.

The composition containing an oily phase may be in the form of a water-in-oil (W/O) or oil-in-water (O/W) emulsion. According to one preferred embodiment, it is in the form of an oil-in-water emulsion.

Tthe compositions of the invention may also contain adjuvants that are known or currently unknown in the cosmetic or dermatological field, such as hydrophilic or lipophilic gelling agents, hydrophilic or lipophilic active agents, preserving agents, antioxidants, solvents, fragrances, fillers, screening agents, bactericides, odour absorbers, dyestuffs and salts. The amounts of these various adjuvants are those that are conventionally used in the field under consideration, and, for example, from 0.01% to 20% of the total weight of the composition, and preferably from 0.01% to 10% of the total weight of the composition. Depending on their nature, these adjuvants may be introduced into the fatty phase, into the aqueous phase and/or into lipid spherules.

As active agents, the composition may contain active agents conventionally used in cosmetics, such as agents for preventing the adhesion of bacteria to the surface of the skin, desquamating agents, moisturizers, anti-seborrhoeic agents, depigmenting or pro-pigmenting agents, anti-glycation agents, NO-synthase inhibitors, 5α-reductase inhibitors, lysyl and/or prolyl hydroxylase inhibitors, agents for stimulating the synthesis of dermal or epidermal macromolecules and/or for preventing their degradation, agents for stimulating fibroblast or keratinocyte proliferation and/or keratinocyte differentiation, muscle relaxants, tensioning agents, anti-pollution agents or free-radical scavengers, calmatives, lipolytic agents or agents with a direct or indirect favourable activity on reducing adipose tissue, agents acting on the capillary circulation, and agents acting on the energy metabolism of cells.

Advantageously, besides phytanetriol, the composition may contain other agents that prevent the adhesion of bacteria to the surface of the skin, such as the oils and fatty substances described in patent application EP-1 313 086, or the alkoxylated plant oils described in patent application FR-2 832 057. It may also advantageously contain other active agents selected from the group consisting of desquamating agents, moisturizers and anti-seborrhoeic agents, and mixtures thereof.

According to another embodiment of the invention, the compositions used may also comprise at least one organic photoprotective agent and/or at least one mineral photoprotective agent active in the UVA and/or UVB range (absorbers), which are water-soluble or liposoluble, or even insoluble in the cosmetic solvents commonly used.

The preferred organic photoprotective agents are selected from the group consisting of ethylhexyl salicylate, ethylhexyl methoxycinnamate, octocrylene, phenylbenzimidazolesulfonic acid, benzophenone-3, benzophenone-4, benzophenone-5,4-methylbenzylidenecamphor, terephthalylidenedicamphorsulfonic acid, disodium phenyldibenzimidazoletetrasulfonate, 2,4,6-tris(diisobutyl 4′-aminobenzalmalonate)-s-triazine, anisotriazine, ethylhexyl triazone, diethylhexylbutamidotriazone, methylenebis(benzotriazolyl)tetramethylbutylphenol and drometrizole trisiloxane, and mixtures thereof.

The mineral photoprotective agents are selected from the group consisting of pigments or nanopigments (mean size of the primary particles: generally between 5 nm and 100 nm and preferably between 10 nm and 50 nm) of coated or uncoated metal oxides, for instance nanopigments of titanium oxide (amorphous or crystallized in rutile and/or anatase form), iron oxide, zinc oxide, zirconium oxide or cerium oxide, which are all UV-photoprotective agents that are well known per se. Standard coating agents are, moreover, alumina and/or aluminium stearate. Such coated or uncoated metal oxide nanopigments are described in particular in patent applications EP 518 772 and EP 518 773.

The photoprotective agents are generally present in the compositions according to the invention in proportions ranging from 0.1% to 20% by weight relative to the total weight of the composition, and preferably ranging from 0.2% to 15% by weight relative to the total weight of the composition.

Gelling agents which may be mentioned, for example, are cellulose derivatives such as hydroxyethylcellulose and alkylhydroxyethylcelluloses such as cetylhydroxyethylcellulose; algal derivatives such as satiagum; natural gums such as tragacanth or guar gum; synthetic polymers such as carboxyvinyl polymers or copolymers and in particular those sold under the names Carbopol^{R} by the company Goodrich or Synthalen^{R} by the company 3V SA. The proportion of gelling agent preferably ranges from 0.1% to 2% relative to the total weight of the composition.

The compositions used according to the invention may be more or less fluid and may have the appearance of a white or coloured cream, an ointment, a milk, a lotion, a serum, a paste or a mousse. They may optionally be applied to the skin in the form of an aerosol. They may also be in solid form and, for example, in the form of a stick.

The compositions used according to the invention are preferably obtained according to a preparation process comprising at least two steps. The first step generally comprises preparing an aqueous dispersion of cubic gel particles as defined above, by fragmentation, using a homogenizer, of compounds as defined above and of water, optionally in the presence of water-insoluble ionic amphiphilic lipids and/or of hydrophilic and/or lipophilic active principles and/or of a dispersing and stabilizing agent, as are defined above. The homogenizer may be of the rotor-stator type with a high shear rate, such as Virtis^{R} or Heidolph Diax 600^{R}, or a high-pressure homogenizer working at between 200 and 1 800 bar approximately (20 to 180 MPa).

Needless to say, it is possible at this stage in the preparation of the aqueous dispersion of cubic gel particles to introduce various additives and/or active principles into the aqueous phase. After formation of the cubic gel particles, the dispersing and stabilizing agent is generally outside the said particles.

The second step then generally comprises adding to the said dispersion obtained an oily phase optionally containing certain lipophilic active principles and/or additives and in subjecting the mixture to a mechanical stirring, which may be carried out in particular using a homogenizer of the same type as those defined above.

Various additives and/or active principles may also be introduced at this stage in the preparation. Moreover, when it is desired to prepare a gelled dispersion, in a third step, an aqueous solution containing a gelling agent is generally added to the mixture obtained after the second step.

The use of phytanetriol according to the invention finds an application more specifically in cosmetics or dermatology. In particular, compositions containing phytanetriol may be used for cleansing and/or removing makeup from and/or caring for the skin. Phytanetriol may also be used in the context of the present invention in antisun products, in makeup products, for instance foundations, lipsticks, mascaras or face powders, and/or in deodorants.

The present invention also relates to a cosmetic process for preventing and/or combating disorders associated with the adhesion of microorganisms, comprising the application to the skin and/or mucous membranes of a composition according to the invention, containing phytanetriol in a physiologically acceptable medium.

More specifically, the invention relates to a non-therapeutic cosmetic process for treating greasy skin, comprising the topical application of a composition according to the invention, containing phytanetriol in a physiologically acceptable medium.

One particular aspect of the invention relates to the non-therapeutic use of phytanetriol, as an agent for preventing or reducing the adhesion of microorganisms to the surface of the skin and/or mucous membranes, in a skin makeup-removing composition.

Thus, another subject of the present invention relates to a process for cleansing and/or removing makeup from the skin, comprising the topical application of a composition containing phytanetriol according to the invention, in a physiologically acceptable medium.

Since the microbial flora of the surface of the skin is responsible for a large number of disorders ranging from simple unpleasantness (odour, spots, etc.) to more serious diseases, a subject of the present invention is also the cosmetic use of phytanetriol, as an agent for reducing unpleasant body odour, and/or for body hygiene.

Another aspect of the invention thus relates to a non-therapeutic cosmetic process for reducing unpleasant body odour, comprising the topical application of a composition according to the invention containing phytanetriol, in a physiologically acceptable medium.

A subject of the present invention is also the use of phytanetriol for the preparation of a dermatological composition for preventing and/or combating acne, more particularly juvenile acne. In particular, the composition contains no antiacne agent other than phytanetriol.

A subject of the present invention is also the use of phytanetriol for the preparation of a dermatological composition for preventing and/or combating mycoses. In particular, the composition contains no antimycotic agent other than phytanetriol.

The term “intended for body hygiene” means a substance intended to be placed in contact with the various surface parts of the human body and/or with mucous membranes, and/or with the teeth, in order to cleanse them, protect them, keep them in good condition, modify their appearance, fragrance them, or correct their odour.

The examples that follow serve to illustrate the invention without, however, being limiting in nature. Depending on the case, the compounds are cited as chemical names or as CTFA names (International Cosmetic Ingredient Dictionary and Handbook).

### EXAMPLE 1

| | | |
|---|---|---|
| Phase A: | Phytanetriol | 2.94% |
| | Monocetyl phosphate | 0.06% |
| | Water | 1.6% |
| Phase B: | Polysorbate 40 | 1% |
| | Glycerol | 4% |
| | Water | 46.15% |
| Phase C: | Sal fat | 2% |
| | Octyldodecanol | 2% |
| | Apricot oil | 6% |
| | Isocetyl stearate | 3% |
| | Cyclohexasiloxane | 6% |
| | UV-screening agent | 1% |
| | Fragrance | 0.2% |
| Phase D: | Hydroxyethylcellulose | 1% |
| | Pentasodium | 0.033% |
| | ethylenediaminetetramethylenephosphonate | |
| | Water | 23.017% |

Procedure:

The mixture of phases A and B was stirred at 60-70° C. until homogenized, and then cooled to 25° C.

Phase C was then heated to about 50° C. and homogenized using a magnetic stirrer, and then cooled to room temperature.

Phase C was then added to the mixture A+B at room temperature with stirring over 15 minutes.

The mixture obtained was then homogenized twice at high pressure (600 bar at room temperature).

The preparation obtained was then gelled using the mixture of phase D (preswollen by blending using a Rayneri mixer at room temperature).

A homogeneous cream was thus obtained, which, after application to the skin, prevents the adhesion of bacteria to the surface of the skin. This cream is suitable for treating mycoses and/or acne.

### EXAMPLE 2

### Microbial Antiadhesion Test

Protocol:

The activity of phytanetriol was demonstrated on reconstructed epidermis.

Before bacterial adhesion, the reconstructed epidermis is placed in contact for 2 hours with the composition of Example 1 at 37° C. 1 ml of bacterial suspension of Staphylococcus aureus at a concentration of 10⁷ microorganisms/ml in Tryptone salt is then added thereto. After incubation for 24 hours at 37° C., the bacterial suspension is emptied out and five rinsings are performed with 1 ml of sterile distilled water. The reconstructed epidermis detached from its support is then ground using a processor in 18 ml of Tryptone salt. This suspension is diluted tenfold in the Tryptone salt, and 1 ml of the dilution is then inoculated in 15 ml of Trypticase soybean agar and incubated for 24 hours at 37° C. The adherent and viable cells are then counted.

This antiadhesion test makes it possible to evaluate the efficacy of molecules alone or of finished products.

Before the antiadhesion test, the following viability test is performed:

A mixture of bacteria/test product, in the same ratio as in the antiadhesion test, is brought into contact for 24 hours at 37° C. The microorganisms are counted by tenfold dilution in Tryptone salt and inoculation, using a scraper, of 100 μl on Trypticase soybean agar. The colonies are counted after incubation for 24 hours at 37° C.

The viability test performed prior to the antiadhesion test makes it possible to set aside any bactericidal component of the molecules or finished products tested, and to demonstrate only the antiadhesive activity.

Results:

The result obtained are summarized in the following table:

| | Activity Antiadhesion | |
|---|---|---|
| Formula tested | Quantitative results | Qualitative results |
| Composition of Example 1 | −1.7 | Good |
| Pure phytanetriol | −1.9 | Good |

The quantitative results correspond to a reduction in the decimal logarithm of the mean number of viable Staphylococcus aureus adherent on the reconstructed epidermis after treatment with the test composition or test molecules, relative to the decimal logarithm of the mean number of viable Staphylococcus aureus adherent on the reconstructed epidermis after treating with water under the same conditions. The results are considered as significant if the difference is greater than 0.5 log.

The qualitative results obtained for the composition and for pure phytanetriol are expressed by a variant term as a function of the values of Log reduction of the adhesion of the microorganisms after 24 hours relative to the same test using water:

| | |
|---|---|
| results better than those obtained with water | Pro-adhesion |
| results identical to those obtained with water | No effect |
| between 0.5 and 1 log reduction relative to water | Poor |
| between 1 and 1.5 log reduction relative to water | Moderate |
| between 1.5 and 2 log reduction relative to water | Good |
| reduction of 2 log or more relative to water | Excellent |

The above written description of the invention provides a manner and process of making and using it such that any person skilled in this art is enabled to make and use the same, this enablement being provided in particular for the subject matter of the appended claims, which make up a part of the original description and including a method for reducing the adhesion of microorganisms to the surface of skin and/or mucous membranes, comprising applying thereto a microorganism adhesion reducing effective amount of phytanetriol to skin and/or mucous membranes in need thereof, the cosmetic use of phytanetriol in a composition as an agent for preventing or reducing the adhesion of microorganisms to the surface of the skin and/or mucous membranes, and the use of phytanetriol for preventing or combating pathologies associated with the adhesion of microorganisms to the surface of the skin and/or mucous membranes. Other embodiments of the invention fully described and enabled include applying the phytanetriol to greasy skin, or areas of the skin and/or mucous membranes subject to unpleasant odors, or to skin comprising acne, or to skin and/or mucous membranes comprising or susceptible to mycoses. In preferred embodiments the microorganism adhesion reducing amount is an amount sufficient to effectively treat or prevent at least one pathology associated with the adhesion of microorganisms to the surface of the skin and/or mucous membranes, such as greasy skin, unpleasant odors, acne, and mycoses.

As used above, the phrases “selected from the group consisting of,” “selected from the group consisting of,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method for reducing the adhesion of microorganisms to the surface of skin and/or mucous membranes, comprising applying thereto a microorganism adhesion reducing effective amount of phytanetriol to skin and/or mucous membranes in need thereof.

2. The method of claim 1, wherein said amount is an amount sufficient for preventing or combating at least one pathology associated with the adhesion of microorganisms to the surface of the skin and/or mucous membranes selected from the group consisting of greasy skin, unpleasant odors, acne, and mycoses.

3. The method of claim 1, wherein the phytanetriol is present in a composition, and is present therein in an amount of from 0.001% to 20% by weight relative to the total weight of the composition.

4. The method of claim 1, wherein the phytanetriol is present in cubic gel particles.

5. The method according to claim 4, wherein the cubic gel particles comprising phytanetriol are in aqueous dispersion.

6. The method according to claim 5, wherein the cubic gel particles are formed by a mixture comprising (i) 0.1% to 15% by weight, relative to the total weight of the composition, of phytanetriol, optionally combined with an N-2-alkoxycarbonyl N-methylglucamine derivative and/or an unsaturated fatty acid monoglyceride, and (ii) 0.05% to 3% by weight, relative to the total weight of the composition, of at least one dispersing and stabilizing agent, the said agent being selected from the group consisting of surfactants that are water-soluble at room temperature, comprising a saturated or unsaturated, linear or branched fatty chain containing from 8 to 22 carbon atoms.

7. The method according to claim 6, wherein the relative weight proportion of compound (i) relative to the weight of the dispersing and stabilizing agent (ii) is between 2 and 200.

8. The method according to claim 6, wherein the N-2-alkoxycarbonyl N-methylglucamine derivatives correspond to formula (I) below: 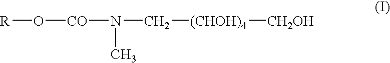
in which R represents a branched alkyl radical containing from 6 to 18 carbon atoms.

9. The method according to claim 8, wherein the N-2-alkoxycarbonyl N-methylglucamine derivative is selected from the group consisting of N-2-hexyldecyloxycarbonyl-N-methylglucamine, N-2-ethylhexyloxycarbonyl-N-methylglucamine, N-2-butyloctyloxycarbonyl-N-methylglucamine, and mixtures thereof.

10. The method according to claim 6, wherein the cubic gel particles comprise, as compound (i), a mixture comprising from 1% to 40% by weight of phytanetriol relative to the weight of the mixture, and from 60% to 99% by weight of N-2-alkoxycarbonyl N-methylglucamine derivative relative to the weight of the mixture.

11. The method according to claim 6, wherein said cubic gel particles comprise an unsaturated fatty acid monoglyceride selected from the group consisting of glyceryl monooleate, glyceryl monolinoleate and mixtures thereof.

12. The method according to claim 6, wherein the cubic gel particles comprise, as compound (i), a mixture comprising 1% to 50% by weight of phytanetriol relative to the weight of the mixture, and from 50% to 99% by weight of unsaturated fatty acid monoglyceride relative to the weight of the mixture.

13. The method according to claim 6, wherein the said dispersing and stabilizing agent is selected from the group consisting of:
(1) alkyl or alkenyl ethers or esters of a polyol,
(2) N-acyl amino acids and derivatives thereof, and peptides N-acylated with an alkyl or alkenyl radical, and salts thereof,
(3) alkyl or alkenyl ether or ester sulfates, derivatives thereof and salts thereof,
(4) polyoxyethylenated fatty alkyl or alkenyl ethers or esters,
(5) polyoxyethylenated alkyl or alkenyl carboxylic acids and salts thereof,
(6) N-alkyl or alkenyl betaines,
(7) alkyl or alkenyl trimethylammoniums and salts thereof, and
(8) mixtures thereof.

14. The method according to claim 4, wherein the cubic gel particles range from 0.05 μm to 1 μm in size.

15. The method according to claim 5, wherein the dispersion of cubic gel particles further comprise at least one water-insoluble ionic amphiphilic lipid.

16. The method according to claim 15, wherein the said water-insoluble ionic amphiphilic lipid is selected from the group consisting of:
(i) phospholipids,
(ii) phosphoric esters of fatty acids,
(iii) water-insoluble N-acyl derivatives of glutamic acid and salts thereof,
(iv) sodium cetyl sulfate,
(v) sodium cocoyl monoglyceride sulfate,
(vi) water-insoluble quaternary ammonium derivatives, and
(vii) mixtures thereof.

17. The method according to claim 4, wherein the particles further comprise at least one hydrophilic and/or lipophilic active principle.

18. The method according to claim 4, wherein the cubic gel particles are present in a composition, and present in an amount ranging from 0.1% to 20% by weight relative to the total weight of the composition.

19. The method according to claim 18, wherein the composition further comprises at least one organic photoprotective agent and/or at least one mineral photoprotective agent active in the UVA and/or UVB range.

20. The method according to claim 1, comprising applying said phytanetriol to greasy skin, or areas of the skin and/or mucous membranes subject to unpleasant odors, or to skin comprising acne, or to skin and/or mucous membranes comprising or susceptible to mycoses.

21. The method according to claim 20, wherein the method comprises applying said phytanetriol to skin comprising acne, and wherein no antiacne agent other than phytanetriol is applied.

22. The method according to claim 20, wherein the method comprises applying said phytanetriol to skin and/or mucous membranes comprising or susceptible to mycoses, and wherein no antimycotic agent other than phytanetriol is applied.

23. The method according to claim 4, wherein the cubic gel particles comprise, as compound (i), phytanetriol and at least one N-2-alkoxycarbonyl N-methylglucamine derivative corresponding to formula (I) below: 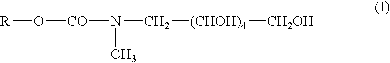
in which R represents a branched alkyl radical containing from 6 to 18 carbon atoms.

24. The method according to claim 23, wherein the N-2-alkoxycarbonyl N-methylglucamine derivative is selected from the group consisting of N-2-hexyldecyloxycarbonyl-N-methylglucamine, N-2-ethylhexyloxycarbonyl-N-methylglucamine and N-2-butyloctyloxycarbonyl-N-methylglucamine, and mixtures thereof.

25. The method of claim 1, wherein said amount is an amount sufficient for preventing or combating at least one pathology associated with the adhesion of microorganisms to the surface of the skin and/or mucous membranes.
